(19) United States
(12) Patent Application Publication
(10) Pub. No.: US 2023/0263255 A1
(43) Pub. Date: Aug. 24, 2023

(54) RUGBY HEADGEAR EVALUATION SYSTEM AND METHOD

(71) Applicant: VIRGINIA TECH INTELLECTUAL PROPERTIES, INC., Blacksburg, VA (US)

(72) Inventors: Steve Rowson, Blacksburg, VA (US); Emily Kieffer, Pittsburg, PA (US); Stefan Duma, Blacksburg, VA (US); Mark Begonia, Christianburg, VA (US)

(21) Appl. No.: 18/172,109

(22) Filed: Feb. 21, 2023

Related U.S. Application Data
(60) Provisional application No. 63/312,211, filed on Feb. 21, 2022.

(51) Int. Cl. A42B 3/04; A61B 5/00

(52) U.S. Cl. CPC A42B 3/0433; A61B 5/6803; A61B 2562/0219

(57) ABSTRACT

Various embodiments relating to methods for evaluating injury mitigation performance of helmets that are used for contact sports (e.g., rugby) are described. In one embodiment, a method for evaluating injury mitigation performance of a helmet includes applying first, second, and third impact configurations to a first helmet. The method further includes generating acceleration and velocity data based on impacts that occur as part of the three impact configurations. The method further includes determining injury risk values based on the generated acceleration and velocity data. The method also includes determining an overall risk metric based on the injury risk values and exposure values.

### CROSS REFERENCE TO RELATED APPLICATIONS

This application claims the benefit of and priority to U.S. Provisional Application Ser. No. 63/312/211, filed Feb. 21, 2022, titled “RUGBY HEADGEAR EVALUATION SYSTEM,” the entire contents of which is hereby incorporated herein by reference.

### BACKGROUND

Head injury is an inevitable risk in any contact sport. In rugby, this risk may be compounded by the fact that players generally wear minimal protective gear compared to players of other contact sports. Concussion is often the most commonly reported head injury in rugby, accounting for a majority of all head injuries. Rugby generally includes physically aggressive moves, such as tackles, which can often induce head trauma for players. Poorly designed headgear may be insufficient in providing protection against concussions and can often give players a false sense of security while playing.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 illustrates a cross sectional view of an example headform that can be used for impact testing of helmets in accordance with various embodiments of the present disclosure.

FIG. 2 illustrates an adapter that can be used to connect the example headform shown in FIG. 1 to a neck structure in accordance with various embodiments of the present disclosure.

FIG. 3 illustrates a side view of the example headform shown in FIG. 1 with the adapter secured to the example headform in accordance with various embodiments of the present disclosure.

FIG. 4A illustrates a pendulum impact testing apparatus in a resting position in accordance with various embodiments of the present disclosure.

FIG. 4B illustrates the pendulum impact testing apparatus impacting the example headform shown in FIG. 1 in accordance with various embodiments of the present disclosure.

FIGS. 5A-5D illustrate close-up views of an impactor striking various locations of a helmet positioned on the example headform shown in FIG. 1 in accordance with various embodiments of the present disclosure.

FIG. 6 is a flowchart illustrating exemplary steps of a method to evaluate concussion mitigation performance of helmets in accordance with various embodiments of the present disclosure.

### DETAILED DESCRIPTION

Aspects of the present disclosure relate to methods for evaluating injury mitigation performance of helmets that are used for contact sports requiring protective head equipment (e.g., rugby). Current rugby evaluation standards are similar to many historical helmet evaluation standards and typically evaluate only a linear acceleration component. However, both linear and angular components of velocity and acceleration occur during head impacts, and it is important to evaluate angular velocity relative to brain injuries as part of impact testing of helmets. By lowering both linear acceleration and angular velocity in laboratory tests, it has been shown that brain injury risk is lowered in real-world head impact events. Given the serious head injuries observed in contact sports, both linear acceleration and angular velocity measures should be analyzed when evaluating the biomechanical performance of helmets for rugby and other contact sports.

According to various embodiments, a testing method can measure or evaluate the concussion mitigation performance of helmets, such as rugby helmets, used for contact sports or other activities. The method is referenced herein as a Summation of Tests for the Analysis of Risk (STAR) method in some examples, but variations of the method can be practiced based on the concepts described herein, regardless of the use of any shorthand names. Although the embodiments of the present disclosure are described in connection with the evaluation of rugby helmets, other types of helmets used for contact sports or similar activities may be evaluated using the systems and methods described herein.

The testing methodology combines impact testing of helmets with an injury risk function as well as exposure data to generate a summary of helmet performance. Impact testing of helmets may be carried out with various dummy headforms that are mounted on various dummy necks. For example, one such configuration can include a rugby helmet positioned on a NOCSAE® headform that has been mounted on a Hybrid III neck. The NOCSAE® headform and the Hybrid III neck correspond to one example configuration, and other configurations involving dummy headforms and necks may be used for impact testing without departing from the scope of the embodiments.

Instrumentation or sensors can be positioned within the headform. The sensors can measure one or more of linear acceleration, angular velocity, angular acceleration, and other inertial measurements. In some cases, angular acceleration may be determined from measured angular velocity. Impact tests can be performed at a range of impact locations and energy levels that include both centric and non-centric impact configurations, which can impact the evaluation of concussion mitigation performance. For each impact test, the peak linear acceleration and angular velocity values are inserted into a brain injury risk function, and the values from the brain injury risk function can be multiplied by an exposure value to obtain a weighted risk value.

The weighted risk values from the impact tests can be further evaluated using a function or equation. The function or equation aggregates the data from the impact tests into a number or metric, as a score representative of the performance of helmets. The score can then be used to categorize helmets into a rating system that includes numerical ranges (e.g., 1-5). In one example, helmets with higher ratings do a better job of managing impact energy and ultimately lowering the linear acceleration and rotational velocity values the head would experience for a given impact, although other rating scales can be relied upon. The rating system can differentiate complex helmet performance into usable information for consumers. On-field studies have shown brain injury reduction rates in athletes who wear higher rated helmets.

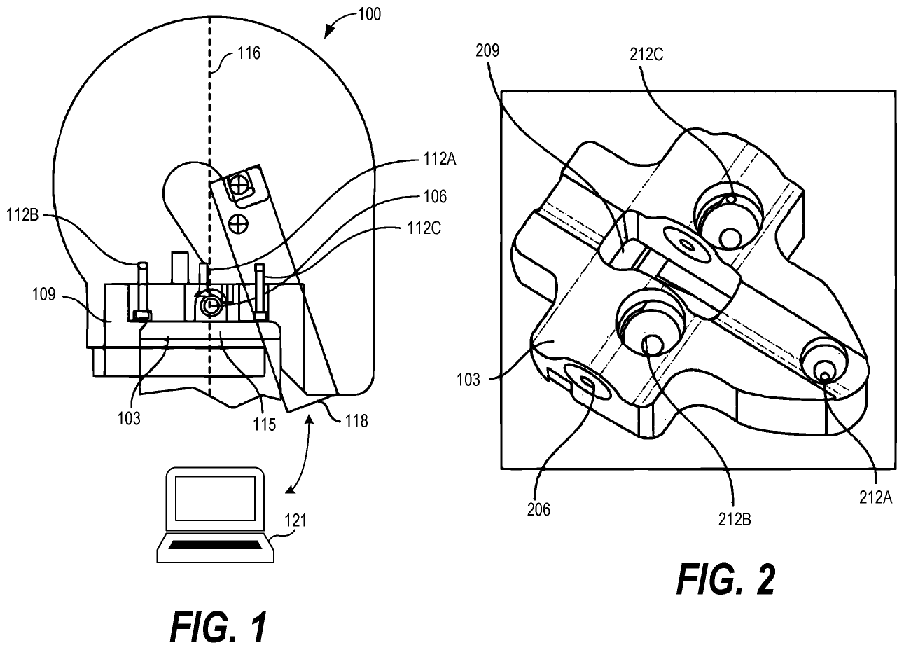

Turning to the drawings, FIG. 1 shows a cross sectional view of a headform 100 that can be used for impact testing of helmets (e.g., rugby helmets) used for contact sports or other activities according to the methods described herein. The headform 100 includes an opening 109 in a lower region (toward the chin of the headform 100), a sensor package 118, fasteners 112A-112C, an occipital condyle pin 106, and an adapter 103.

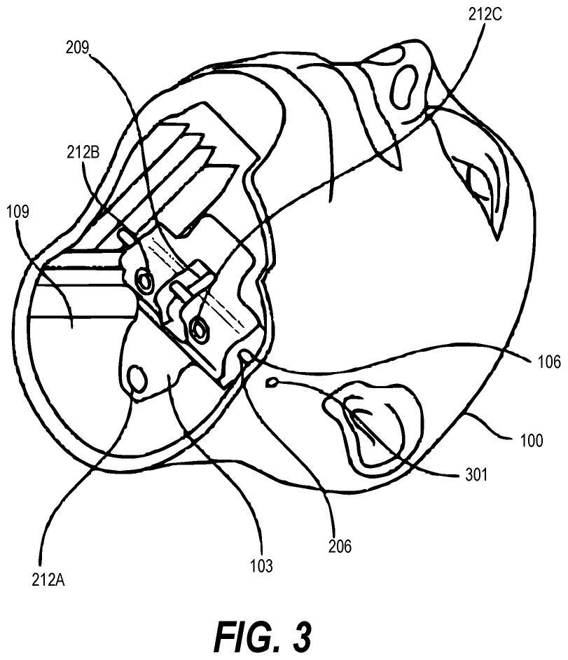

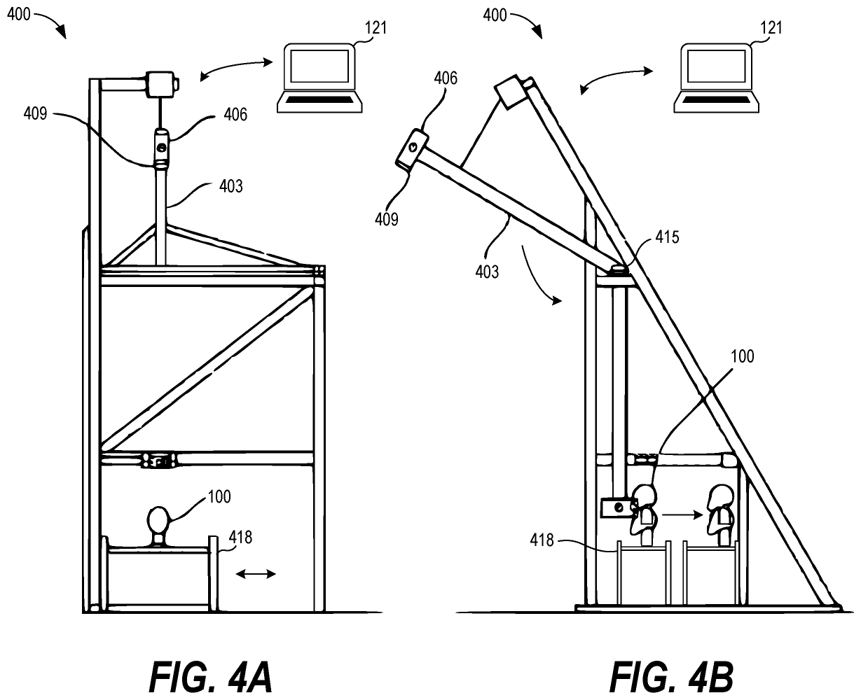

FIG. 1 also illustrates a portion of a neck 115 that attaches to the headform 100 through the adapter 103 at the opening 109. The adapter 103 can be used to mount the headform 100 to the neck 115. In this respect, the adapter 103 can include a custom adapter plate that provides anatomically accurate relative locations of the occipital condyle pin 106 and center of gravity 116 of the headform 100. In some embodiments, the headform 100 and the neck 115 can include a NOCSAE® headform and a Hybrid III 50^{th} percentile neck, respectively. However, other combinations of dummy headforms and necks can be used to conduct the impact tests discussed herein. A detailed view of the adapter 103 is illustrated in FIG. 2. A side view of the headform 100 with the attached adapter 103 is illustrated in FIG. 3.

The adapter 103 can include mounting holes 212A-212C, which can receive fasteners 112A-112C located within the headform 100, to secure the headform 100 to the adapter 103. As such, the adapter 103 can improve the anatomical accuracy of the location of the center of gravity 116 of the headform 100. A bore 206 in the adapter 103 and opening 301 (shown in FIG. 3) in the headform 100 can be matched to receive the occipital condyle pin 106. An opening 209 in the adapter 103 can be used to connect the neck 115 to the headform 100. The adapter 103, through the mounting holes 212A-212C and the fasteners 112A-112C, enable the headform 100 to be secured in a position that allows impact testing of helmets (e.g., rugby helmets) used for contact sports or related activities. The headform 100 can include various types of dummy headforms, such as the NOCSAE® headform, that are suitable for fitting rugby helmets. The neck 115 can include various types of dummy necks, such as the Hybrid III 50^{th} percentile male neck, that can be attached to the headform 100 and is suitable for impact testing of helmets.

The sensor package 118 can be attached near the center of gravity 116 of the headform 100. The sensor package 118 can include one or more accelerometers, angular rate sensors, or other inertial measurement or sensor units that measure linear acceleration, angular velocity, angular acceleration, and other inertial metrics generated by head impacts during testing of helmets, such as rugby helmets. In some embodiments, the sensor package 118 can include a six degree of freedom (6DoF) sensor package that includes three accelerometers and a triaxial angular rate sensor. However, other combinations of linear accelerometers, angular accelerometers, and angular rate sensors can be employed within the headform 100 to measure linear acceleration, angular velocity, and/or angular acceleration. In some cases, angular acceleration values may be determined based on the obtained angular velocity data.

The sensor package 118 can be embodied as one or more accelerometers. As one example, the sensor package 118 is capable of measuring acceleration (i.e., the rate of change of velocity) as compared to its own instantaneous rest frame and provide feedback signals or data representative of the acceleration. The accelerometers of the sensor package 118 can be single or multi-axis accelerometers, capable of detecting both the magnitude and the direction of the acceleration in some cases, as a vector quantity. In some cases, the sensor package 118 can be an inertial measurement unit (IMU) capable of also measuring orientation, positional angular information, velocity, and other inertial information related to the headform 100. Thus, the sensor package 118 can also sense orientation, coordinate acceleration, vibration, shock, and falling motions in some cases. Examples of the accelerometers of the sensor package 118 can include accelerometers from Endevco®, Piezotronics®, Dytran®, Honeywell®, Bosch®, and other manufacturers.

The sensor package 118 can be communicatively coupled with computing device 121 for data transfer using any suitable wired or wireless interface. The computing device 121 can include one or more processing circuits, for example, having processors and memories or memory devices, which can be coupled to an interface for data communication. The processing circuits of the computing device 121 can process data, as described herein, such as linear acceleration data, angular velocity data, angular acceleration data, and other types of data. In some cases, the computing device 121 can include data sampling, filtering, and processing devices or systems, for processing data received from the sensor package 118. The computing device 121 can also include power sources, such as batteries or other power sources. The local interface of the computing device 121 can be embodied as one or more wired, wireless, or wired and wireless local interfaces. The sensor package 118 may communicate with the computing device 121 through one or more wired, WiFi, Bluetooth®, near-field communication (NFC), radio-frequency identification (RFID), wireless infrared, ultra-wideband, wireless induction, long range (LoRa), Z-Wave®, ZigBee®, etc., interfaces.

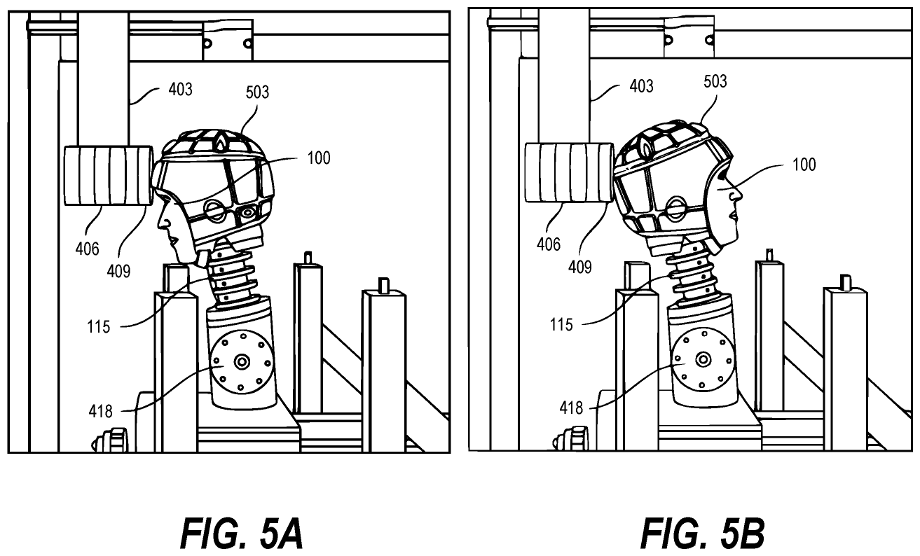

FIGS. 4A-4B illustrate various views of a pendulum impact testing apparatus 400 that can be used to impact the headform 100 during impact testing of helmets. As discussed previously, the testing methods described herein incorporate impact testing of helmets and use of a concussion risk function to analyze linear acceleration and rotational velocity values resulting from the impact tests. The pendulum impact testing apparatus 400, as illustrated in FIGS. 4A-4B and described below, is provided as a representative example. Other impact testing apparatuses can be used to implement the evaluation approaches described herein. For example, the shapes, sizes, and weights of the testing apparatus 400 can vary as compared to those described below.

The pendulum impact testing apparatus 400 includes a movable arm 403, an impactor 406, an impactor face 409, a pivot point 415, and a sliding mass 418. The pendulum impact testing apparatus 400 can be used to conduct the impact testing portion of the testing methods. Use of the pendulum impact testing apparatus 400 is beneficial in that it enables an easily repeatable system to conduct the impact tests.

In one example, the movable arm 403 can be embodied as a 10.16×5.8 cm rectangular aluminum tubing, with the impactor 406 having a mass of 15.5 kg. The length of the arm 403 from the center of the pivot point 415 to the center of the impactor 406 can be 190.5 cm. The arm 403 can have a total mass of 37 kg and a moment of inertia of 72 kg·m², 78% of which is the impacting mass. The impactor face 409 can be embodied as a CELL-FLEX® vinyl nitrile 740 foam and is 12.7 cm in diameter and 1.27 cm in thickness in one example. The pendulum impact testing apparatus 400 can be controlled by a winch system equipped with an electromagnet attached to the arm 403 in order to raise and release it at desired angles correlated with various velocities. In some cases, the winch system may be pneumatically or hydraulicly driven. Other pendulum impact testing apparatuses and related impact testing tools can be relied upon to gather impact data for evaluation using the concussion risk functions described herein.

In addition, components of the impact testing apparatus 400 may be controlled or directed, at least in part, by the computing device 121. For example, the computing device 121 may be in data communication with the winch system to control the angle and velocity at which the impactor 406 impacts a rugby helmet on the headform 100 using electromechanical actuators, switches, motors and other systems. The impact testing apparatus 400 may communicate with the computing device 121 through one or more wired, WiFi, Bluetooth®, near-field communication (NFC), radio-frequency identification (RFID), wireless infrared, ultra-wideband, wireless induction, long range (LoRa), Z-Wave®, ZigBee®, etc., interfaces. According to one embodiment, the pendulum impact testing apparatus 400 can be configured so that the impactor face 409 strikes the helmet on the headform 100 between 3.0-4.5 m/s.

In order to carry out the impact tests, a helmet is first positioned on the headform 100. As discussed previously, the headform 100 and the neck 115 can include a NOCSAE® headform and a Hybrid III 50^{th} percentile male neck, respectively. The NOCSAE® headform can be mounted to the Hybrid III 50^{th} percentile male neck using the adapter 103, which provides anatomically accurate relative locations of the occipital condyle pin 106 and the center of gravity 116 of the headform 100. The headform 100 and the neck 115 are then positioned on the sliding mass 418. The sliding mass 418 can be designed to simulate the effective torso mass of a 50^{th} percentile male during a head impact according to one example, thereby increasing the biofidelity of the impact configuration. In one example, the sliding mass 418 can be mounted to an adjustable table commonly used for impact testing.

The impactor 406 provides an impacting mass that is adjustable to emulate a full range of head impact characteristics experienced by individuals, such as participants in rugby or other contact sports. In this regard, the impactor face 409 may be of a certain shape, size, and weight that emulates the aforementioned characteristics of objects rugby participants may frequently come into contact with to cause injury. In some cases, the impactor face 409 may be curved to emulate the shape of a curved object. When activated, the arm 403 pivots from the pivot point 415 causing the impactor face 409 to strike the helmet on the headform 100.

FIG. 4A illustrates the pendulum impact testing apparatus 400 in its resting position before activation. The arm 403 has not yet been activated, and the headform 100 positioned on the sliding mass 418 is in its starting position. FIG. 4B illustrates the pendulum impact testing apparatus 400 that has been activated. In some cases, the pendulum impact testing apparatus 400 can be controlled by the computing device 121, allowing for the velocity and angle of impact to be configured in an automated fashion based on control of a user interface of the computing device 121. In some cases, the pendulum impact testing apparatus 400 may be manually configured by a user (e.g., user who controls the winch system). When the arm 403 pivots from the pivot point 415 and the impactor surface 409 strikes the helmet on the headform 100, the sliding mass 418 and the headform 100 may change positions as depicted.

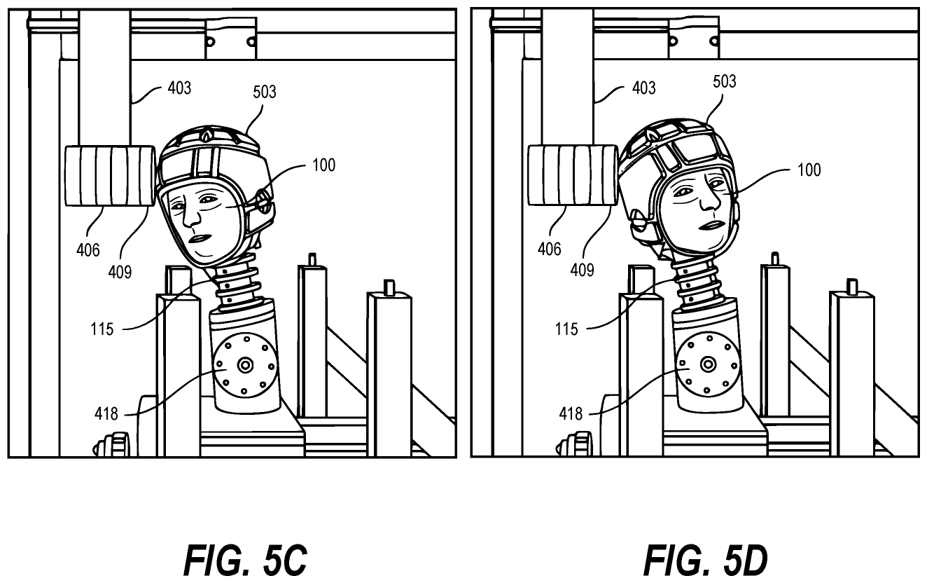

FIGS. 5A-5D illustrate a close-up view of the impactor face 409 striking various locations of a helmet 503 positioned on the headform 100. The helmet 503 can include any helmet that is used for contact sports or related activities. In the illustrations shown, the helmet 503 is depicted as a rugby helmet.

The methods described herein rely upon impact tests conducted at multiple locations on the helmet 503. In one embodiment, three or more impact configurations can be used to test the model of the helmet 503 for a total of twelve impact tests. These configurations can be defined as impacts at four impact locations of the helmet 503 at three different impact velocities. The four different impact locations include a front of the helmet 503, a front boss of the helmet 503, a rear boss of the helmet 503, and a rear of the helmet 503. The front and the rear of the helmet 503 are illustrated in FIGS. 5A and 5B, and the front boss and the rear boss of the helmet 503 are illustrated in FIGS. 5C and 5D. The front boss location of the helmet 503 refers to a location between the front of the helmet 503 and a side of the helmet 503, and the rear boss location of the helmet 503 refers to a location between the rear of the helmet 503 and a side of the helmet 503.

The three different impact velocities are configurable by the user. The impact velocities were selected as 3.0 m/s, 3.6 m/s, and 4.5 m/s, in one example, for testing of rugby helmets to replicate low, medium, and high energy impacts according to one experiment that was conducted. The 3.0 m/s velocity is representative of the 90^{th} percentile of the on-field head impact data for rugby, the 3.6 m/s velocity is representative of the 95^{th} percentile, and the 4.5 m/s is representative of the 99^{th} percentile. Other impact velocities can be relied upon in some cases.

To generate accurate test results, each impact location of the helmet 503 is impacted the same number of times. That is, applying the three impact configurations to the helmet 503 can include a total of twelve impacts (i.e., four impacts at a first impact velocity, four impacts at a second impact velocity, and four impacts at a third impact velocity). Thus, the helmet 503 can be impacted at the aforementioned four locations the same number of times but at different velocities by the impactor face 409. Each impact location being impacted only once may ensure that any deformation caused by testing does not affect any future tests.

In a different example, each impact location of the helmet 503 may be impacted more than once to generate more sample data. Generating more sample data may improve reproducibility of the tests and improve accuracy, although at the cost of some deformation to the helmet 503 caused by testing. For example, each of the three impact configurations may be repeated so that each impact location is impacted twice at the three different impact velocities mentioned above. Repeating the three impact configurations for a total of two impacts at each location of the helmet 503 can result in approximately 24 tests.

In another example, the three impact configurations may be applied to a separate sample of the same model helmet as the helmet 503. For instance, a second helmet of the same model as the helmet 503 may be used for testing, and each of the three impact configurations at the mentioned four locations (i.e., four impacts at a first impact velocity, four impacts at a second impact velocity, and four impacts at a third impact velocity) can be applied to the second helmet. Additionally, the three impact configurations may be repeated on the second helmet similar to the impact tests of the first helmet. In such a scenario, 24 tests may be carried out for each sample to generate a total of 48 tests.

The linear acceleration and angular velocity measurements gathered from the 48 tests can be compared to a control set of linear acceleration and angular velocity measurements obtained from tests to a NOCSAE headform (at the aforementioned four impact locations at the three different velocities) not equipped with any protective headgear. A comparison of test results to a control set of test results can establish a baseline rating score of a model of the helmet 503 being tested and additional rating scales that discern concussion risk reduction improvements of the helmet 503 beyond the baseline rating score.

With the impact tests occurring at multiple locations on the helmet 503, FIGS. 5A-5D depict the impactor face 409 impacting the front of the helmet 503, the rear of the helmet 503, the front boss of the helmet 503, and the rear boss of the helmet 503, respectively. When the helmet 503 is impacted, resultant linear acceleration and angular velocity measurements are generated by the sensor package 118 based on translations and rotations of the headform 100 on the sliding mass 418. Data representative of the resultant linear acceleration and angular velocity measurements that is generated by the sensor package 118 is provided to the computing device 121, in one example, and the computing device 121 calculates a concussion risk value for each impact based on a concussion risk function described below. The concussion risk value(s) are then used to determine a concussion risk metric for the helmet 503. Although the concussion risk value or values can be calculated by the computing device 121, concussion risk values can also be calculated manually by an individual in some cases based on the data generated by the sensor package 118.

The impactor face 409 may be flat, curved, or of a different shape to simulate the surface of the impacting medium the tests are designed to replicate. However, other impactor surfaces of a different shape, weight, and surface can be used to simulate other injury contact surfaces that may be prevalent in different contact sport. In some embodiments, fewer or greater than three impact configurations may be used to conduct impact testing of the helmet 503. That is, any one of the three aforementioned impact locations may or may not be impacted, and a second helmet of the same model as the helmet 503 may or may not be used. In some cases, more than two helmets of the same model may be tested.

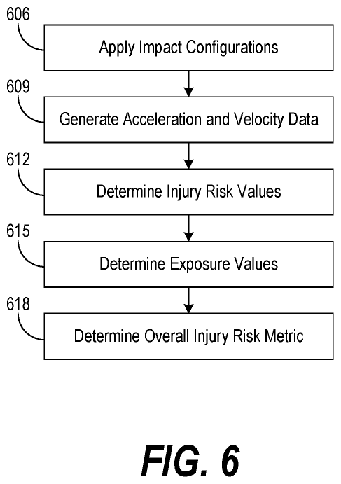

However, each impact location being impacted an equal number of times is an important consideration that is factored in when applying the concussion risk function, which will be discussed in detail in the following paragraphs with respect to the flowchart shown in FIG. 6. According to one example, each impact location can be considered contributing equally (25% each for four impact locations) based on impact testing performance when analyzing the concussion mitigation performance of the helmets described herein. In other examples, the data gathered for each impact location can be weighted and considered unequally (e.g., with a different weight given to each impact location).

FIG. 6 is a flowchart outlining the steps of a method for evaluating the concussion mitigation performance of helmets, such as rugby helmets, used in contact sports or similar activities. At step 606, a set of impact configurations are applied to a helmet that is selected for testing. For example, referring back to FIGS. 4A-4B, the helmet 503 is selected for testing of concussion mitigation performance. The helmet 503, which can include various rugby helmets, is positioned on the headform 100 based on the helmet manufacturer's fitting guidelines. Chinstrap and laces (if applicable) are tightened for best fit according to manufacturer instructions. The headform 100 can be mounted to the neck 115 using the adapter 103. The headform 100 and the neck 115 can be mounted to the sliding mass 418 and can be positioned within an impact area of the pendulum impact testing apparatus 400.

After the helmet 503 is positioned on the headform 100 and mounted to the testing apparatus, the method can include applying a first impact configuration to the helmet 503 with a first impact velocity. For instance, the first impact configuration can include impacts to multiple locations on the helmet 503 with the first impact velocity. In accordance with one example, as shown in FIGS. 5A-5D, the pendulum impact testing apparatus 400 and the headform 100 can be configured so that the impactor 406 impacts the front of the helmet 503, the rear of the helmet 503, the front boss of the helmet 503, and the rear boss of the helmet 503 for a total of four impact locations. However, other locations of the helmet 503 may be impacted in some cases. In some cases, the pendulum impact testing apparatus 400 can be controlled by a winch system equipped with an electromagnet in order to raise and release the movable arm 403 at desired angles corresponding to a desired impact velocity. In some cases, the winch system may be pneumatically or hydraulicly driven. In some cases, the pendulum impact testing apparatus 400 may also be controlled by the computing device 121.

The method can also include applying a second impact configuration to the helmet 503 with a second impact velocity and applying a third impact configuration to the helmet 503 with a third impact velocity. The second and third impact configurations can include impacts to the same locations as the first impact configuration but at different velocities. For example, the first impact configuration can include impacts to the aforementioned four locations at 3.0 m/s, the second impact configuration can include impacts to the aforementioned four locations at 3.6 m/s, and the third impact configuration can include impacts to the aforementioned four locations at 4.5 m/s. However, other velocities may be relied upon in certain cases.

In some cases, it may be beneficial to impact each impact location of the helmet 503 more than once to generate more sample data. Generating more sample data may improve reproducibility of the tests and improve accuracy of the sample data, although at the cost of some deformation to the helmet 503 caused by testing. For example, each of the three impact configurations may be repeated so that each impact location is impacted twice at the three different impact velocities mentioned above. Repeating the three impact configurations for a total of two impacts at each location of the helmet 503 can result in 24 tests.

In another example, the three impact configurations (i.e., impacting the aforementioned four locations at three different velocities) may be applied to a separate sample of a same model helmet as the helmet 503. For instance, a second helmet of the same model as the helmet 503 may be used additionally for testing, and each of the three impact configurations at the mentioned four locations (i.e., four impacts at a first impact velocity, four impacts at a second impact velocity, and four impacts at a third impact velocity) can be applied to the second helmet. Additionally, the three impact configurations may be repeated on the second helmet as well so an equal number of data samples are generated for each of the two samples being tested. If each impact location is tested twice for the helmet 503 and the second helmet, a total of 48 tests may be conducted with 24 tests for each sample helmet.

Different impact velocities are selected for each of the three impact configurations to replicate low, medium, and high energy impacts. For example, a velocity of 3.0 m/s may be selected as the impact velocity for one of the impact configurations as replicating a low energy impact. In addition, a velocity of 3.6 m/s may be selected as the impact velocity for one of the impact configurations as replicating a medium energy impact. In addition, a velocity of 4.5 m/s be selected as the impact velocity for one of the impact configurations as replicating a high energy impact. The 3.0 m/s velocity is representative of the 90^{th} percentile of the on-field head impact data, the 3.6 m/s velocity is representative of the 95^{th} percentile, and the 4.5 m/s is representative of the 99^{th} percentile according to one example.

Other impact velocities can also be relied upon. In some cases, a velocity of greater than 4.5 m/s may be selected to replicate a high energy impact, and a velocity of lower than 3.0 m/s may be selected to replicate a low energy impact. If the three impact configurations are applied to a second helmet of a like model as the helmet 503 and each impact location is tested twice, a total test matrix of 48 tests can occur. Table 1, below, provides example headform translations and rotations that can occur after each impact at the aforementioned four impact locations:

TABLE 1
Headform Translations and Rotations on the Sliding
Mass 418 for each Test Condition.

| Location | Y (cm) | Z (cm) | Ry (deg) | Rz (deg) |
|---|---|---|---|---|
| Front | 0 | +3.0 | −10° | 0° |
| Front Boss | −3.5 | +7.0 | −15° | −60° |
| Rear Boss | +2.0 | +6.0 | −5° | −120° |
| Rear | 0 | +5.0 | −15° | 180° |

All measurements above were made using the SAE J211 coordinate system in relation to a “zero” condition in which the headform 100 was in a position of 0° Y and Z-axis rotation and the median (midsagittal) and basic (transverse) plane intersection of the headform 100 was aligned with the center of the impactor 406. The x-position was set such that the head just touches the impactor face 409 when the pendulum arm 403 is in a neutral vertical position for each location.

Additionally, at step 606, the method can include applying the first, second, and third impact configurations to the headform 100 without the helmet 503. The impacts can be performed in a similar way, using the same velocities and impact locations, as performed with the helmet 503, on the bare headform 100. Data from the bear-headform impacts are used in Equation 2, as described below.

In step 609, the method includes generating acceleration and related inertial data based on the application of the impact configurations at step 606. The acceleration data can be generated by various accelerometers and sensors that can be positioned within a headform that is being used for impact testing. The generation of the acceleration and related inertial data can occur during the application of the impact configurations at step 606. The generation can occur for the headform 100 with the helmet 503, the headform 100 with a second helmet of the same model as the helmet 503, and with the bare headform 100. For example, the sensor package 118, which is positioned within the headform 100 near the center of gravity 116, may include accelerometers, angular rate sensors, and other IMUs, as described above. In one embodiment, the sensor package 118 includes a six degree of freedom (6DoF) sensor package that includes three accelerometers and a triaxial angular rate sensor. The three accelerometers can measure linear acceleration data, and the triaxial angular rate sensor may measure angular velocity data. In some embodiments, the sensor package 118 may also include angular accelerometers configured to measure angular acceleration, among other accelerometers and/or angular rate sensors.

Thus, for each impact that occurs at the aforementioned impact locations, the sensor package 118 can generate linear acceleration data, angular velocity data, angular acceleration data, and other inertial measurement data. The computing device 121 may receive the generated data as part of step 609, including the acceleration data and angular velocity data, among other inertial measurement data, and process it as described herein. In cases where angular acceleration is not measured, the computing device 121 may determine the angular acceleration values based on differentiating the angular velocity data.

According to one example, the acceleration data and angular velocity data measured for each of the 48 impacts were sampled at 20,000 Hz and filtered using a 4-pole Butterworth low pass filter according to SAE J211 (Instrumentation for Impact Test), with a cutoff frequency of 1650 Hz (CFC 1000) for the accelerometer data and 256 Hz (CFC 155) for the angular rate sensor data. The sampling and filtering can be performed by processing circuitry of the sensor package 118, by the computing device 121, or by intermediate processing circuitry between the sensor package 118 and the computing device 121. Angular acceleration data values can also be determined by differentiating the angular rate data by the computing device 121 in some cases. Resultant values can also be calculated for linear acceleration (e.g., in g-force) and angular velocity (rad/sec) by the computing device 121.

Moving to step 612, the method includes determining injury risk values associated with the first, the second, and the third impact configurations. Each injury risk value can be associated with the probability of an injury, such as the probability of a concussion. The computing device 121 can process the linear acceleration and angular velocity values generated in step 609 to determine the injury risk values. For example, for each impact that occurs with respect to the first, the second, and the third impact configurations discussed in step 606, the computing device 121 is configured to calculate an injury risk value based on the following function or equation:

$$R ( a , ω ) = \frac{1}{1 + {e}^{- ( - 10.2 + 0.0433 + a + 0.19686 * ω - 0.0002075 * a ω )}} \qquad ( Eq . 1 )$$

This equation, also referenced herein as the injury risk function or concussion risk function, outputs an injury risk value R based on the resultant linear acceleration (α) and resultant angular velocity (ω) data associated with each impact. The injury risk function accounts for both linear acceleration and angular velocity data because they are both correlated and predictive of an injury, such as a concussion. The injury risk function in Equation 1 is based on an adaptation of a multivariate logistic regression analysis of instrumented football player data paired with diagnosed concussions. Thus, the injury risk function accounts for the types of linear and rotational peak acceleration values known to be associated with brain injuries. The multivariate logistic regression analysis can be used to model risk as a function of both linear and angular head acceleration. To modify the risk function in Equation 1, other estimated linear relationships between rotational velocity and acceleration can be used to replace the rotational acceleration terms. Using the injury risk function enhances the data analysis by increasing the importance of higher acceleration impacts.

Moving to step 615, the method includes determining exposure values. An exposure value can be determined for each impact location (e.g., front, rear, front boss, rear boss, etc.) that occurs as part of the overall impact test. The exposure values are used to weight the relative frequency that an individual, such as a rugby player, would experience an impact at one of the impact locations. Thus, in one example, the exposure values are related to a distribution matching on-field head impact data collected from collegiate rugby players.

In one example, the exposure values can be selected or determined so that each impact location contributes equally to the overall injury or concussion risk metric, described below, if the helmet 503 performs the same at each impact location during the impact tests discussed in steps 606 and 609. However, the exposure values can also be altered or modified to account for differences in the impact responses due to the non-isotropic nature of the Hybrid II neck and the variance of the impact location relative to the center of gravity of the head. Table 2 lists example exposure values E used for each location/velocity combination based on a 25% contribution for each impact location.

TABLE 2
Exposure Values Used for each Location/Velocity
Combination to Obtain Weighted Concussion Risk Values.

| Location | 3.0 m/s | 3.6 m/s | 4.5 m/s |
|---|---|---|---|
| Front | 20.0 | 0.7 | 0.6 |
| Front Boss | 26.9 | 1.5 | 1.1 |
| Rear Boss | 19.1 | 1.1 | 0.6 |
| Rear | 26.1 | 1.5 | 0.8 |

In step 618, the method includes determining an overall injury risk metric based on the injury risk values R from step 612 and the exposure values E from step 615. The computing device 121 can determine or calculate the overall injury risk metric using the injury risk values and the exposure values. In one example, the computing device 121 can determine or calculate the overall injury risk metric based on the equation listed below:

$$STAR = \frac{{\sum }_{L = 1}^{4} {( {\sum }_{V = 1}^{3} E ( L , V ) * R ( A , ω ) )}_{HG}}{{\sum }_{L = 1}^{4} {( {\sum }_{V = 1}^{3} E ( L , V ) * R ( A , ω ) )}_{BARE}} , \qquad ( Eq . 2 )$$

where E represents an exposure value for an impact, L represents an impact location, V represents an impact velocity for an impact, and R represents an injury risk value for an impact. With reference to the three impact configurations discussed in step 606, individual injury risk values for each of the impacts are multiplied by corresponding exposure values in Equation 2. The multiplied concussion risk values and exposure values, also known as weighted concussion risk values, are also summated together to generate an overall score (e.g., STAR score) for the helmet 503 being tested in Equation 2. The overall score for the helmet 503 is compared to (e.g., divided by) a control score obtained from tests to the bare headform 100 (at the same impact locations at the three different velocities) not equipped with any protective headgear. The computing device 121 may process or evaluate the data related to the E, L, V, and R values to determine the STAR score for a given helmet.

The STAR score for a given helmet model is then used to determine a corresponding rating or STAR rating in some cases. The STAR score is different from the STAR rating. The STAR rating may range up to five stars for the best available helmets. The STAR rating thresholds are determined based on the average STAR scores of a tested helmet. For example, a STAR value of 0.5 represents a 50% reduction in concussion incidence of the impacts tested. A STAR value of 1 represents identical performance to the bare headed condition.

TABLE 3
Thresholds to Match STAR Values to a STAR Rating

| STAR | |
|---|---|
| Value | Number of Stars |
| <0.3 | 5 |
| <0.4 | 4 |
| <0.5 | 3 |
| < 0.6 | 2 |
| ≥0.6 | 1 |

Rugby helmets, as with other helmets or protective headgear, should reduce the head impact accelerations to potentially reduce the number of injuries in contact sports such as rugby. A limitation of the impacts discussed for the three impact configurations can be that that only one size of helmet may be tested. Such testing conditions assume that performance is consistent throughout each size of helmet. However, there still could be deviation in performance as size increases or decreases due to potential changes in padding configuration and thickness. Accordingly, further embodiments of the present disclosure may include testing helmets of different sizes and/or weight, with the helmets being the same model. Additional embodiments may include testing helmets designated for different sexes (e.g., male or female) of the same model.

Many researchers have been using lab head impact data alongside computer models that simulate brain tissue strain. The linear and angular head acceleration and velocity data discussed herein may be utilized with computer models. This would allow for a better understanding of rugby head injury response specifically in relation to brain deformation. Computer modeling has also been used in helmet research to design optimized helmet prototypes. These techniques could be applied to the rugby head injury mechanisms to develop a helmet that is able to substantially reduce head injury risk.

The flowchart of FIG. 6 shows an exemplary implementation of the methods described herein, as applied to testing for concussion mitigation performance of helmets. Although the flowchart of FIG. 6 shows a specific order of execution, it is understood that the order of execution may differ from that which is depicted. For example, the order of execution of two or more blocks may be scrambled relative to the order shown. Also, two or more blocks shown in succession in FIG. 6 may be executed concurrently or with partial concurrence. Further, in some embodiments, one or more of the blocks shown in FIG. 6 may be skipped or omitted. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present disclosure.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, at least the following is claimed:

1. A method of evaluating a helmet, comprising:
applying a first impact configuration to the helmet with a first impact velocity, the first impact configuration comprising impacts to a front of the helmet, a front boss of the helmet, a rear boss of the helmet, and a rear of the helmet at the first impact velocity;
applying a second impact configuration to the helmet with a second impact velocity, the second impact configuration comprising impacts to the front of the helmet, the front boss of the helmet, the rear boss of the helmet, and the rear of the helmet at the second impact velocity;
generating a plurality of linear acceleration and angular velocity values associated with the first impact configuration and the second impact configuration, the plurality of linear acceleration and angular velocity values comprising a respective linear acceleration value and angular acceleration value for each impact of the first impact configuration and the second impact configuration;
determining a plurality of injury risk values associated with the first impact configuration and the second impact configuration based on the plurality of linear acceleration and angular velocity values; and
determining an overall injury risk metric for the helmet based on the plurality of injury risk values and a plurality of exposure values.

2. The method of claim 1, further comprising positioning the helmet on a headform, the headform being mounted on a neck using an adaptor to locate a center of gravity of the headform.

3. The method of claim 2, further comprising mounting the headform on a sliding mass that simulates an effective torso mass of a human during a head impact, the sliding mass being mounted to an adjustable table.

4. The method of claim 2, wherein the plurality of linear acceleration and angular velocity values are generated by a six degrees of freedom (6DoF) sensor package located near the center of gravity.

5. The method of claim 4, wherein the 6DoF sensor package comprises three accelerometers and a triaxial angular rate sensor.

6. The method of claim 1, wherein the first velocity and the second velocity range from 3 meters per second (m/s) to 4.5 m/s.

7. The method of claim 1, wherein the first impact configuration and the second impact configuration are applied using a pendulum impactor.

8. The method of claim 7, wherein the pendulum impactor comprises an arm, a pivot point, and an impactor surface.

9. The method of claim 1, further comprising categorizing the overall injury metric into a rating system, the rating system comprising a numerical range with a plurality of numerical threshold levels.

10. The method of claim 1, further comprising applying a third impact configuration to the helmet with a third impact velocity, the third impact configuration comprising impacts to the front of the helmet, the front boss of the helmet, the rear boss of the helmet, and the rear of the helmet at the third impact velocity.

11. The method of claim 1, further comprising:
applying the first impact configuration and the second impact configuration to a bare headform; and
determining a plurality of injury risk values associated with the bare headform, wherein
determining the overall injury risk metric for the helmet comprises determining the overall injury risk metric based further on the plurality of injury risk values associated with the bare headform.

12. The method of claim 1, wherein the helmet comprises a rugby helmet.

13. A method of evaluating a helmet, comprising:
applying an impact configuration to the helmet on a headform with a first impact velocity, a second impact velocity, and a third impact velocity;
applying the impact configuration to the headform with the first impact velocity, the second impact velocity, and the third impact velocity;
generating helmet acceleration data associated with the impact configuration to the helmet;
generating headform acceleration data associated with the impact configuration to the headform;
determining a plurality of injury risk values based on the helmet acceleration data and the headform acceleration data; and
determining an overall injury risk metric for the helmet based on the plurality of injury risk values.

14. The method of claim 12, further comprising determining a plurality of exposure values to weight a relative frequency that an impact would occur at an impact location on the helmet.

15. The method of claim 14, wherein determining the overall injury risk metric for the helmet comprises determining the overall injury risk metric based further on the plurality of exposure values.

16. The method of claim 13, wherein the plurality of exposure values are weighted so that each impact location of the impact configuration contributes equally in the overall injury risk metric.

17. The method of claim 13, wherein the helmet comprises a rugby helmet.

18. A method of evaluating a helmet, comprising:
applying a first impact configuration to the helmet and a second helmet with a first impact velocity;
applying a second impact configuration to the helmet and the second helmet with a second impact velocity;
applying a third impact configuration to the helmet and the second helmet with a third impact velocity;
generating a plurality of linear acceleration and angular velocity values associated with the first, the second, and the third impact configurations;
determining a plurality of injury risk values associated with the first, the second, and the third impact configurations;
determining a plurality of exposure values associated with the first, the second, and the third impact configurations; and
determining an overall injury risk metric based on the plurality of injury risk values and the plurality of exposure values. helmet.

19. The method of claim 18, wherein the second helmet is a same model as the

20. The method of claim 19, wherein the helmet and the second helmet comprise rugby helmets.
